(19) United States
(12) Patent Application Publication
(10) Pub. No.: US 2026/0191520 A1
(43) Pub. Date: Jul. 9, 2026

(54) REVERSIBLE EXPANDABLE PUPIL EXPANDER

(71) Applicant: Med Invent Devices Pvt. Ltd., Kolkata (IN)

(72) Inventor: Suven BHATTACHARJEE, Kolkata (IN)

(73) Assignee: Med Invent Devices Pvt. Ltd., Kolkata (IN)

(21) Appl. No.: 19/555,272

(22) Filed: Mar. 3, 2026

(51) Int. Cl. A61B 17/02; A61B 17/00

(52) U.S. Cl. CPC A61B 17/0231; A61B 17/0293; A61B 2017/00862; A61B 17/0218

(57) ABSTRACT

A pupil expansion device for ophthalmic surgery includes a closed-loop ring body defining a central aperture and a plurality of flanges arranged circumferentially around the ring body. The flanges comprise non-folded flanges defining a plane of the device and folded flanges positioned alternately between the non-folded flanges. The folded flanges are connected to the ring body by notch regions and are movable between a resting configuration and an expanded configuration to enlarge the pupil. In one embodiment, the non-folded flanges, folded flanges, and notch regions are coplanar in the resting configuration. In another embodiment, the folded flanges are tilted posteriorly relative to the plane defined by the non-folded flanges by an angle between approximately 1 degree and 30 degrees. The device may be formed as a single-piece structure and is configured for use during ophthalmic surgical procedures, including manual small incision cataract surgery.

### BACKGROUND

### Technical Field

The present disclosure relates to ophthalmic surgical devices and, more particularly, to a mechanically expandable, polygonal pupil expansion device with alternate folded flanges that reversibly unfold to provide a temporary enlarged aperture during cataract or anterior segment surgery, including manual small incision cataract surgery (MSICS).

### Background Art

Small pupil management is a critical challenge in cataract surgery and other ophthalmic procedures. Existing pupil expansion devices, such as ring-based expanders and notched iris engagement devices, are generally configured to maintain the pupil in a fixed enlarged configuration once deployed. Such devices do not provide dynamic, surgeon-controlled reversible expansion, temporary enlargement beyond a resting geometry, or alternating structural elements having distinct mechanical behaviours, such as folded and non-folded flanges.

Applicant's earlier disclosures (e.g., U.S. Pat. No. 10,080,558; IN 294336; and EP 2961329) disclose polygonal ring devices with notches and flanges configured to engage and stabilize the pupillary margin. These devices include continuous or discontinuous polygonal ring bodies, notch regions for pupillary engagement, flange structures for iris stabilization, manufacturable forms with or without joints, and positioning features such as openings for instrument engagement.

While such devices provide effective pupil stabilization, they are generally configured to maintain a fixed aperture once engaged and do not disclose reversible elastic enlargement mechanisms or configurations in which alternating flanges assume differing structural states during use. Further, such devices do not disclose configurations in which alternate flanges are oriented out of the plane of the device, including flanges that are tilted relative to a plane defined by adjacent flanges.

Accordingly, there remains a need for pupil expansion devices that permit controlled, temporary enlargement beyond a resting configuration during a surgical manoeuvre, while allowing the device to return automatically toward a compact configuration without removal or replacement. Therefore, embodiments of the present disclosure provide reversible enlargement, enable alternating folded and non-folded flanges, facilitate safe MSICS nucleus prolapse and ensure automatic return to resting geometry.

A pupil expansion device comprising a continuous polygonal ring having eight flanges and eight notches arranged alternately, wherein every alternate flange is a folded flange configured to unfold elastically under radial forces and return automatically to the folded configuration.

### SUMMARY

The disclosure provides a polygonal pupil expansion device, which is preferably octagonal, having eight flanges and eight notches arranged in an alternating sequence. Every alternate flange is a folded flange, and the remaining are non-folded. The folded flanges configured to adopt a first folded configuration in the resting state, adopt a second unfolded configuration under radial traction or engagement forces, and automatically return to the folded configuration due to elastic memory of the material.

The resting internal diameter of the central aperture is approximately 8.0 mm, and the expanded diameter is approximately 8.75 mm, enabling safe prolapse of a large nucleus during MSICS without requiring additional mechanical dilation.

The folded flanges may include internal holes or tabs to facilitate manipulation with microsurgical instruments. The expansion and contraction are reversible, requiring no joints, and preserving structural integrity of the continuous or jointless ring.

The disclosure introduces a new class of dynamic, reversible, surgeon-controlled pupil expanders that integrate seamlessly with standard surgical workflow.

### BRIEF DESCRIPTION OF THE DRAWINGS

FIG. 1 is a perspective view of the pupil expansion device in a resting configuration, showing the folded flanges in their inwardly deflected state;

FIG. 2 is a perspective view of the device in an expanded configuration, illustrating the unfolded flanges and enlarged internal aperture;

FIG. 3 is a top plan view of the device in the resting configuration, defining a central aperture of approximately 8.0 mm;

FIG. 4 is a top plan view of the device in the expanded configuration, defining an enlarged central aperture of approximately 8.75 mm;

FIG. 5 is a composite top plan view showing the resting configuration in solid lines and the expanded configuration in dashed lines, illustrating the reversible enlargement resulting from flange unfolding;

FIG. 6 is an enlarged detail view of a folded flange in the resting configuration, illustrating the hinge region and tab hole;

FIG. 7 is an enlarged detail view of an unfolded flange in the expanded configuration, showing the hinge region in its extended form;

FIG. 8 is a sequential illustration showing a non-dilating pupil, the device in a resting configuration prior to engagement, and the pupil expanded while the device remains in the resting configuration;

FIG. 9 is a method illustration showing temporary enlargement of the device during MSICS nucleus prolapse, wherein the folded flanges unfold elastically to accommodate passage of the nucleus; and

FIG. 10 illustrates a partial perspective view of a variant of the pupil expansion device, showing folded flanges tilted posteriorly relative to a plane defined by non-folded flanges.

### DETAILED DESCRIPTION

The following detailed description is provided to illustrate embodiments of the disclosure and is not intended to limit its scope. Structural features described with reference to one embodiment may be incorporated into any other embodiment unless explicitly stated otherwise.

### Overview of the Device

The pupil expansion device 10 comprises a continuous ring body 12 configured to be positioned at the pupillary margin during ophthalmic surgery. The ring body includes a plurality of circumferentially arranged flanges and notches configured to engage the iris and radially expand the pupil. The device operates in at least two selectable configurations: (a) a resting configuration, in which alternating flanges are folded inward, defining a nominal aperture size of approximately 8.0 mm, and (b) an expanded configuration, in which the folded flanges unfold elastically, increasing the aperture to approximately 8.75 mm or greater.

The device may be formed from a flexible, elastomeric, biocompatible material such as polyimide, PEBAX, PMMA, or other suitable polymeric or metallic materials that permit reversible deformation at ophthalmic force levels.

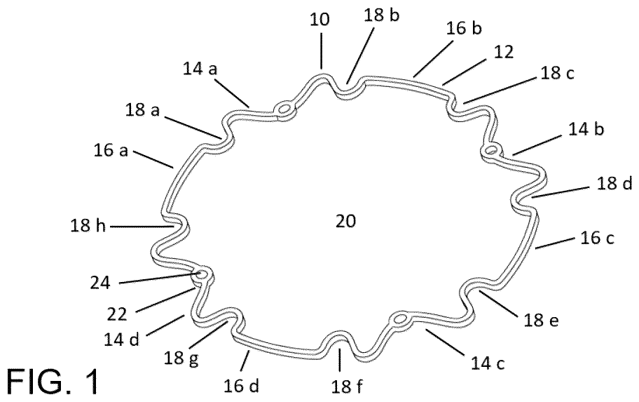

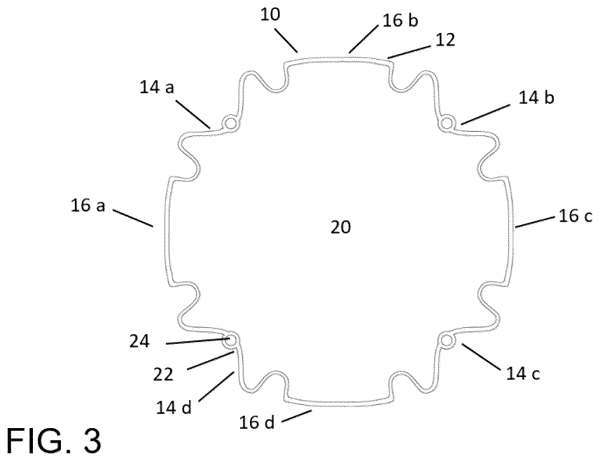

### Resting Configuration (FIGS. 1 and 3)

In the resting configuration shown in FIGS. 1 and 3, the device includes alternating folded flanges 14a, 14b, 14c and 14d and non-folded flanges 16a, 16b, 16c and 16d arranged around the ring body 12 with a resting aperture 20. Each folded flange includes a hinge region 22 formed by a localized reduction in material thickness, curvature modification, or modified cross-section, permitting the flange to bend inward relative to the plane of the adjacent portions of the ring.

The inwardly folded flanges 14a, 14b, 14c and 14d reduce the effective radial extension of the ring, resulting in a nominal pupil expansion diameter of approximately 8.0 mm when the notches 18a, 18b, 18c, 18d, 18e, 18f, 18g and 18h engage the pupillary margin. Each notch includes an outwardly opening entry region and an inwardly directed bulbous receptacle configured to receive and retain a portion of the pupillary margin during engagement. The device is introduced into the anterior chamber in this resting configuration, allowing ease of insertion and atraumatic engagement with the iris.

One or more flanges include tab holes 24, configured to receive microsurgical instruments such as forceps, micro-hooks, or manipulators. These holes facilitate controlled placement, rotation, or removal of the device.

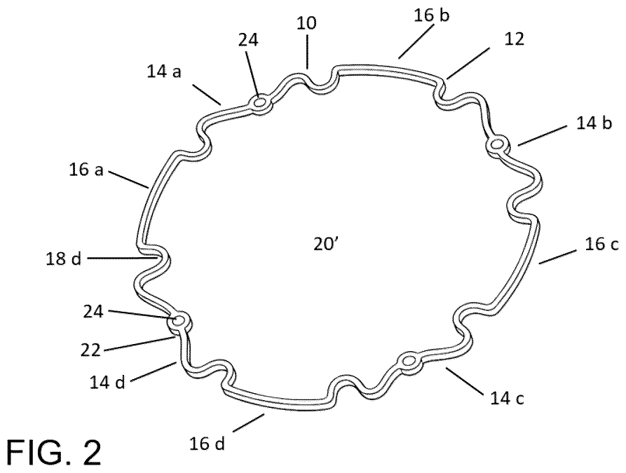

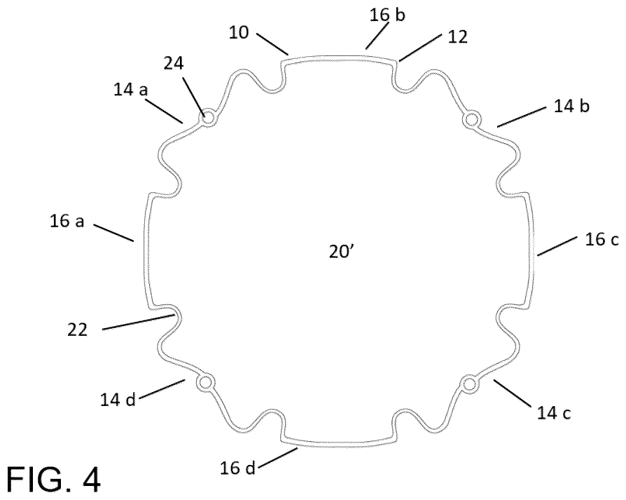

### Expanded Configuration (FIGS. 2 and 4)

FIGS. 2 and 4 illustrate the device in the expanded configuration, achieved when radial forces—such as those produced during MSICS nucleus prolapse—cause the folded flanges 14a, 14b, 14c and 14d to unfold. In this condition, the hinge regions 22 straighten, enlarging the aperture 20.

The unfolding of alternating flanges increases the circumferential span of the ring body 12 and enlarges the central aperture to approximately 8.75 mm. This temporary enlargement facilitates surgical steps requiring increased access, particularly passage of a voluminous lens nucleus into the anterior chamber.

After the deforming force is removed, the material elasticity of the hinge region causes the flanges to automatically return to their folded posture, restoring the resting-state device size.

Upon application of radially outward forces by a surgical instrument, the folded flanges elastically unfold to temporarily enlarge the pupil, and upon release of such forces the device returns toward its resting configuration.

As used herein, “surgeon-controlled reversible expansion” refers to controlled, elastic deformation of the device between a resting configuration and a temporarily enlarged configuration in response to applied forces, followed by automatic return toward the resting configuration upon removal of such forces, and does not refer merely to manual positioning, placement, or manipulation of the device as a whole.

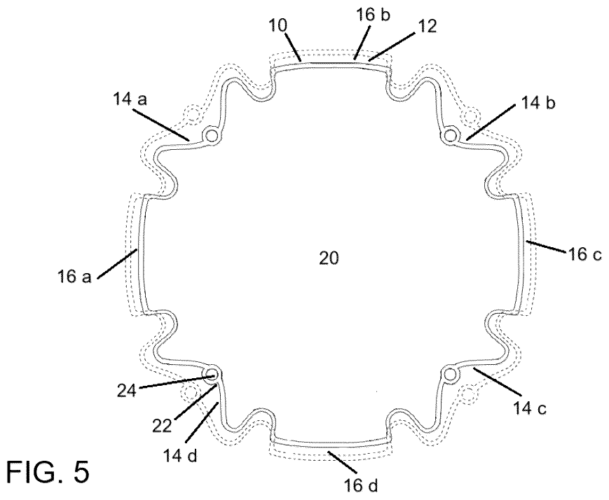

### Comparison of Configurations (FIG. 5)

FIG. 5 is a composite representation showing the resting configuration in solid lines and the expanded configuration in dashed lines. The solid outline represents the device in the resting configuration, while the dashed outline represents the device in the expanded configuration. The figure illustrates the reversible enlargement that occurs as the folded flanges unfold. The dashed traces represent the outward displacement of the flanges, notches, and tab holes during expansion.

This figure emphasizes the asymmetric, alternating nature of the fold/unfold mechanism and visually demonstrates the controlled, limited radial deformation required to transition between aperture diameters.

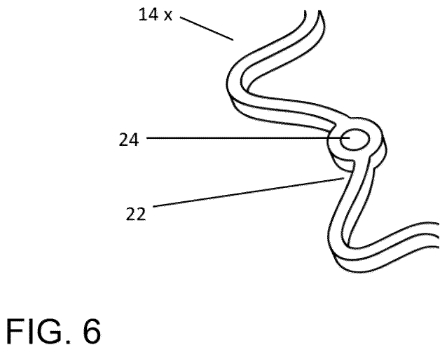

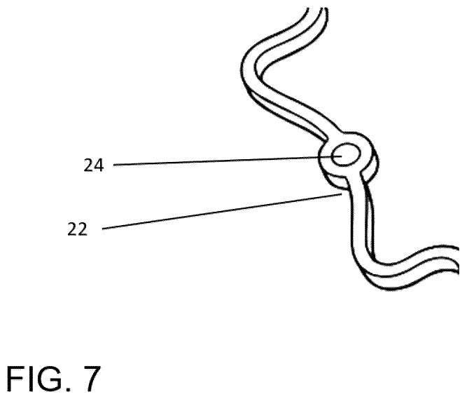

### Folded and Unfolded Flange Structure (FIGS. 6 and 7)

In FIGS. 6 and 7, a representative folded or unfolded flange is illustrated and may be referred to generically as flange 14x, corresponding to any of the folded flanges described elsewhere herein.

FIG. 6 illustrates the geometry of a folded flange 14x in the resting configuration. The hinge region 22 enables localized bending, and the tab hole 24 is accessible for manipulation. The folded contour shortens the radial reach of the flange, reducing the resting aperture.

FIG. 7 illustrates the same flange in the unfolded state, achieved during expansion. The hinge region 22 straightens, permitting maximum radial extension. The transition between FIGS. 6 and 7 represents the essential functional behavior of the disclosure.

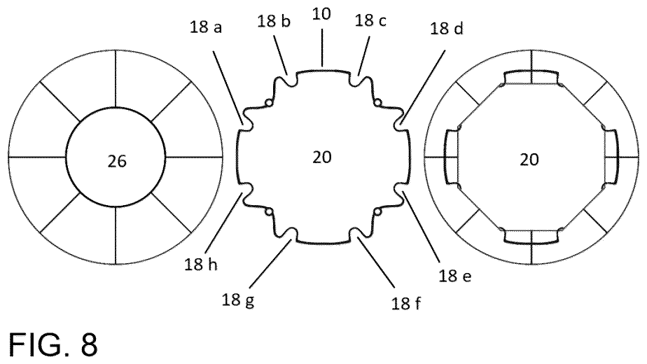

### Pupil Engagement and Resting-State Expansion (FIG. 8)

FIG. 8 demonstrates a sequential method:
1. A non-dilating pupil 26 is shown without the device.
2. The device 10 in its resting configuration is positioned adjacent the pupillary margin.
3. When the notches 18a, 18b, 18c, 18d, 18e, 18f, 18g and 18h engage the iris, the pupil expands to the diameter defined by the resting aperture 20.

Importantly, the device produces clinically significant dilation even without flange unfolding, enabling stable visualization during early surgical steps.

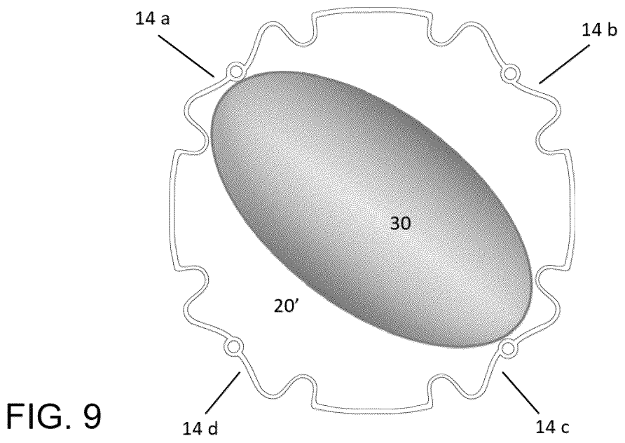

### Temporary Enlargement During MSICS (FIG. 9)

FIG. 9 illustrates the device during nucleus prolapse in MSICS. A cataractous lens nucleus 30 passes anteriorly through the ring body 12. As the nucleus exerts outward radial force on alternating flanges, the folded flanges 14a, 14b, 14c and 14d temporarily unfold, enlarging the aperture 20.

This reversible deformation: increases working space, reduces risk of iris trauma, and avoids the need for alternative dilation devices.

Upon completion of nucleus delivery, the hinge regions 22 elastically restore the flanges to the resting configuration, re-establishing the aperture defined in FIG. 3.

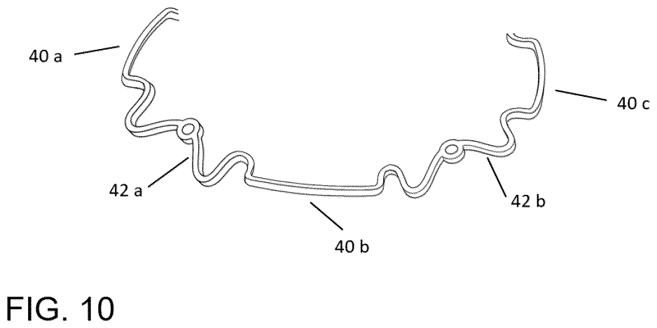

FIG. 10 illustrates an alternative embodiment in which the non-folded flanges 40a, 40b and 40c define a primary plane of the device, while the folded flanges 42a and 42b are inclined posteriorly relative to the plane of the device. The posterior inclination occurs at or adjacent to notch regions connecting the folded flanges to the ring body. Each folded flange may be inclined by an angle in the range of approximately 1 degree to 30 degrees relative to the plane of the device. The inclined configuration may be integrally formed during manufacture or elastically assumed during use, while remaining structurally and functionally consistent with the embodiments of FIGS. 1-9.

While embodiments having eight flanges and eight notches arranged alternately around the ring body are illustrated, the number of folded flanges, non-folded flanges, and notch regions may be varied. In alternative embodiments, the device may comprise at least four flanges and at least four notches, with at least two non-folded flanges defining a plane of the device and at least two folded flanges arranged alternately therewith. Such variations preserve the alternating folded and non-folded flange configuration and reversible expansion functionality described herein.

### Materials and Manufacturing

Devices according to the present disclosure may be manufactured using a variety of material removal or forming techniques. In some embodiments, the device is formed by laser cutting from a polymeric sheet, particularly for configurations in which the folded and non-folded flanges are coplanar in a resting state. In other embodiments, including embodiments in which one or more folded flanges are oriented out of the plane of the device or are tilted relative to a plane defined by the non-folded flanges, the device may be formed by laser forming, laser cutting from a polymeric block, or by other suitable laser-based or non-laser-based forming or shaping processes like molding, stamping, micro-machining, or additive manufacturing. The selection of a manufacturing technique may depend on the desired geometry, material properties, and angular orientation of the folded flanges, and does not limit the structure or function of the device as claimed.

The hinge region 22 may be produced by localized thinning, thermal programming, geometric contouring, or material selection that provides controlled elastic bias toward the folded state.

### Use in Ophthalmic Surgery

The device is compatible with: MSICS, Phacoemulsification, Femtosecond LASER assisted cataract surgery (FLACS), and Any procedure requiring pupil expansion.

The reversible expansion mechanism offers advantages over prior devices by providing two pupil sizes on demand, without requiring removal or replacement of the device.

What is claimed is:

1. A reversible pupil expansion device configured for placement at a pupillary margin, the reversible pupil expansion device comprising:
a resilient ring body defining a central aperture; and
at least four flanges and at least four notches arranged alternately around a perimeter of the ring body, wherein
at least two of the flanges are non-folded flanges defining a plane of the device,
at least two other flanges are folded flanges that are folded radially inward toward the central aperture in a resting state,
the notches open radially outward away from the central aperture, and each of the notches comprises an outward opening and an inward bulbous receptacle configured to engage a pupillary margin,
each folded flange is configured to adopt a folded configuration in the resting state and an unfolded configuration in response to radially outward forces, and
the folded flanges are elastically biased to automatically return to the folded configuration upon release of the forces, thereby reversibly enlarging the central aperture.

2. The device of claim 1, wherein
the folded flanges and the non-folded flanges are coplanar in the resting state.

3. The device of claim 1, wherein
the non-folded flanges remain substantially coplanar during expansion of the device.

4. The device of claim 1, wherein
unfolding of the folded flanges increases a diameter of the central aperture from a first diameter to a second, larger diameter.

5. The device of claim 4, wherein
the first diameter is approximately 8.0 mm and the second diameter is approximately 8.75 mm.

6. The device of claim 1, wherein
the device comprises six flanges or eight flanges arranged alternately with corresponding notches.

7. The device of claim 1, wherein
the ring body is polygonal or octagonal in plan view.

8. The device of claim 1, wherein
each of the folded flanges includes a tab portion having an opening configured for engagement by microsurgical forceps.

9. The device of claim 1, wherein
the folded flanges are connected to adjacent flanges by the notches.

10. The device of claim 1, wherein
the folded flanges are tilted posteriorly relative to the plane defined by the non-folded flanges by an angle between 1 degree and 30 degrees.

11. The device of claim 1, wherein
each of the folded flanges includes a hinge region.

12. The device of claim 11, wherein
the hinge region comprises a reduced-thickness portion or an integrally formed living hinge.

13. The device of claim 1, wherein
the device is configured as a single-piece structure by laser cutting from a polymeric sheet.

14. The device of claim 1, wherein
the folded flanges automatically return to the folded configuration after completion of a surgical manoeuvre.

15. A method of expanding a pupil during ophthalmic surgery, the method comprising:
positioning the device of claim 1 at a pupillary margin and applying radially outward forces to unfold the folded flanges.

16. The method of claim 15, wherein
the pupil is temporarily enlarged beyond a resting configuration.

17. The method of claim 15, wherein
the surgery is manual small incision cataract surgery (MSICS).

18. The method of claim 17, wherein
the pupil is temporarily enlarged during prolapse of a lens nucleus.

19. A pupil expansion system comprising:
the device of claim 1; and
a microsurgical instrument configured to engage the folded flanges via tab openings.

20. The device of claim 1, wherein
the ring body is flexible and resilient.
